(19) United States
(12) Patent Application Publication
(10) Pub. No.: US 2026/0151325 A1
(43) Pub. Date: Jun. 4, 2026

(54) Natural Facial Lotion with Buchu

(71) Applicant: Jo Feltman, Temperance, MI (US)

(72) Inventor: Jo Feltman, Temperance, MI (US)

(21) Appl. No.: 18/968,193

(22) Filed: Dec. 4, 2024

Related U.S. Application Data
(60) Provisional application No. 63/595,418, filed on Nov. 2, 2023.

(51) Int. Cl. A61K 8/9789; A61Q 19/00

(52) U.S. Cl. CPC A61K 8/9789; A61Q 19/00; A61K 2800/5922; A61K 2800/70

(57) ABSTRACT

The present invention relates to a natural facial lotion comprising buchu extract (Agathosma Betulina) as a key ingredient, along a method for its preparation. The buchu plant is indigenous to the Western Cape of South Africa, known for its antibacterial, antimicrobial, and anti-inflammatory properties, making it highly effective for skincare applications. The lotion is formulated to moisturize the skin, even out skin tone, and promote clear, healthy skin. The composition includes additional functional ingredients such as base oils, aloe vera, glycerin and alpha hydroxy acids, to enhance its efficacy and stability.

### BACKGROUND OF THE INVENTION

The present invention relates to a natural facial lotion formulated with buchu extract and a method for its preparation. The buchu plant (Agathosma Betulina) is a native plant found in the Western Cape of South Africa, known for its antibacterial, antimicrobial, and anti-inflammatory properties, making it an excellent candidate for skincare products. The invention leverages the benefits of buchu to create a moisturizing lotion that evens skin tone and promotes clear skin.

### Classification: A61K8/062 OR A61Q19/00 OR A61K8/04

### SUMMARY OF THE INVENTION

The present invention is a novel composition for a facial lotion with the primary ingredient being buchu extract. The formulation comprises buchu infusion, glycerin, glycolic acid or AHA Fruit Acid Complex (inci: water and vaccinium myrtillus fruit/leaf extract and Saccharum officinarum extract, citrus aurantium dulcis fruit extract and citrus limon fruit extract and acer saccharum extract), various beneficial oils such as Grapeseed Oil and Raspberry Seed Oil, Xanthan Gum as a functional stabilizer, Shea Butter, Cetearyl Olivate and Sorbitan Olivate or a compatible emulsifier, tocopherol for mold prevention and skin benefits, essential oils like geranium and lavender, along with natural preservatives to prevent bacterial growth, aloe vera, and other potential future ingredients designed to benefit the skin. The invention also includes a specific method for combining these ingredients in various proportions to create the facial lotion.

### DETAILED DESCRIPTION OF THE INVENTION

The buchu facial lotion comprises the following ingredients and their functions:
Buchu Extract or Buchu Hydrosol: Sourced from the Agathosma Betulina plant, this extract provides antibacterial, antimicrobial, and anti-inflammatory properties to the lotion.
Glycerin: Adds moisturizing properties to the lotion.
AHA Fruit Acid Complex or Glycolic Acid: AHA acts as a natural exfoliant to clear pores, even skin tone, and boost collagen production.
Various Oils: Including but not limited to Grapeseed oil, Raspberry Seed oil, and other beneficial oils, similar to nutrient rich oils and botanical oils.
Xanthan Gum: Serves as a functional stabilizer and thickener.
Shea Butter: Enhances the lotion's texture and moisturizing capabilities.
OliveM 1000 INCI: Cetearyl Olivate and Sorbitan Olivate: or a compatible emulsifier: Facilitates the blending of ingredients.
Tocopherol: Prevents mold growth and offers additional skin benefits.
Essential Oils: Such as geranium or lavender for fragrance and potential therapeutic effects.
Natural Preservative: Prevents bacterial growth, ensuring product longevity.
Aloe Vera INCI: Aloe Barbadensis Leaf Extract: Provides additional skin benefits.

The proportions of these ingredients may vary with a specific range to achieve the desired effects.

### Advantages of the Invention:

Provides a natural and effective solution for facial skincare.
Utilizes the antibacterial properties of buchu extract.
Offers moisturization, even skin tone, and clear skin.
May incorporate additional beneficial ingredients in the future.

### Detailed Description of the Method:

1. Create a buchu infusion using buchu plant and distilled water.
2. Combine ingredients in specified proportions to create the lotion.
3. Ensure thorough mixing and homogeneity.
4. Package the lotion in suitable containers.

### Conclusion:

The Buchu Facial Lotion and its formulation method provide a unique solution to skincare, capitalizing on the natural benefits of buchu extract and a blend of complimentary ingredients. The invention is versatile and can adapt to include other components that enhance skin health.

1: A facial lotion composition comprising an effective amount of buchu extract/hydrosol (Agathosma Betulina) for providing antibacterial, antimicrobial, and anti-inflammatory benefits to the skin.

2: The facial lotion composition of claim 1, further comprising glycerin, aloe vera, glycolic acid or AHA, xanthan gum, shea butter, natural emulsifier, base oils, comprising but not limited to grapeseed oil, raspberry seed oils, tocopherol, essential oils, and a natural preservative, wherein the combination enhances skin hydration, collagen production, and pore-clearing effects.

3: A method of preparing a facial lotion composition, comprising; (a) claim 1 combining the infusion with aloe vera, and natural Alpha Hydroxy Acids (AHA) to help improve moisturization to ingredients, (b) combine glycerin and xanthan gum in a beaker and add to above ingredients, (c) combine and heat shea butter and base oils, (d) blending the (e) mixture with a functional emulsifier; (f) stir in essential oils, tocopherol and natural preservatives to form a stable lotion.

4: The facial lotion composition of claim 1; wherein the formulation comprises:
a. buchu extract/hydrosol in an amount ranging from 30-70%;
b. aloe vera in an amount ranging from 3-10%;
c. glycerin in an amount ranging from 3-10%;
d. cetearyl olivate and sorbitan olivate in an amount ranging from 5-10%;
e. xanthan gum in an amount ranging from .25-1%;
f. essential oils, including but not limited to geranium, in an amount ranging from .20-1%;
g. shea butter in an amount ranging from 3-12%;
h. Alpha Hydroxy Acids (AHA) or glycolic in an amount from 1-10% by weight, with the remainder comprising additional oils, stabilizers, emulsifiers, preservatives, and other functional additives;
i. tocopherol in an amount ranging from .25-1%;
j. base oils, comprising but not limited to grapeseed oil, raspberry seed oil and other plant-derived oils, in an amount ranging from 3-20%.

5. The facial lotion composition of claim 4, wherein other ingredients may be substituted or added in the future to enhance the formulation while preserving its antibacterial, anti-inflammatory, and skin-regenerating properties, provided that such additions do not alter the primary function of the lotion as a skin care product.
